(19) United States
(12) Patent Application Publication
(10) Pub. No.: US 2002/0065443 A1
(43) Pub. Date: May 30, 2002

(54) Process for the nonoxidative dehydrogenation of an alkylaromatic feed stream

(75) Inventors: David L. Williams, Louisville, KY (US); Dennis J. Smith, Louisville, KY (US); Andrzej Rokicki, Prospect, KY (US)

Correspondence Address: Scott R. Cox, Suite 2200, 400 West Market Street, Louisville, KY, 40202, US

(73) Assignee: Sud-Chemie Inc., Louisville, KY (US)

(21) Appl. No.: 09/727,036

(22) Filed: Nov. 30, 2000

(51) Int. Cl. C07C 2/64; C07C 4/06; C07C 5/42

(52) U.S. Cl. 585/444; 585/440; 585/921; 585/926; 585/924; 585/445

(57) ABSTRACT

A process for the nonoxidative dehydrogenation of an alkylaromatic feed stream wherein the feed stream is passed through a radial reactor containing nonoxidative dehydrogenation catalysts, wherein the nonoxidative dehydrogenation catalysts are arranged in vertically layered beds within the radial reactor, and wherein the nonoxidative dehydrogenation catalysts include at least a first and a second nonoxidative dehydrogenation catalyst, wherein at least one of the nonoxidative dehydrogenation catalysts has a different performance and/or operating characteristic than at least one of the other nonoxidative dehydrogenation catalysts.

### CROSS REFERENCE TO RELATED APPLICATIONS

None

### BACKGROUND OF INVENTION

1. Field of Invention

The field of art to which this invention pertains is a process for the nonoxidative dehydrogenation of an alkylaromatic feed stream. More particularly, this invention relates to a process for the nonoxidative dehydrogenation of an alkylaromatic feed stream in a radial reactor utilizing at least two different nonoxidative dehydrogenation catalysts arranged in vertical layers within a vertically layered catalyst bed wherein at least two of the nonoxidative dehydrogenation catalysts have different operating or physical characteristics.

2. Description of Related Art

The dehydrogenation of hydrocarbons is well described in the prior art with both acyclic and aromatic hydrocarbons being converted to correspondingly less saturated products. One of the best known of these dehydrogenation processes is the conversion of ethylbenzene to styrene. Thus, it is well known in the art to react ethylbenzene over a dehydrogenation catalyst, such as iron oxide, at elevated temperature to form styrene. For example, a process for the dehydrogenation of ethylbenzene to styrene is disclosed in U.S. Pat. No. 6,096,937.

The commercial process for the conversion of ethylbenzene to styrene is normally conducted in a series of radial, adiabatic reactors. These reactors generally are elongated, cylindrical, vertical structures which may be very large, ranging in diameter from 5 to 30 feet or more and in length from 10 to about 100 feet or more. Examples of these radial flow reactors are disclosed in the drawings associated with JP 49039971 and 49039972. A process and apparatus of this type for the conversion of ethylbenzene is disclosed in U.S. Pat. Nos. 5,358,698 and 4,039,601. Another vertical reactor used for a different type of chemical reaction is disclosed in U.S. Pat. No. 4,687,656.

It has been recognized that a reactor system containing multiple reactors may produce a higher degree of conversion and may have greater selectivity than is exhibited by a single reactor. Thus, sometimes three or more radial adiabatic reactors are arranged in a serial flow orientation with reheat means located between the reactors to add heat to the reaction.

The normal construction of a radial reactor allows for the input of the ethylbenzene feed stream through an inlet located in the center of the radial reactor. The catalyst is contained within a vertical bed in that reactor occupying a cone-shaped space, which is located outside of a central core of the reactor. The ethylbenzene feed stream enters the reactor through an inlet pipe, then flows radially outward through the catalyst in the vertical bed to an open, annular space which is formed outside of the catalyst bed within the reactor and ultimately the feed stream flows to an outlet for the catalyzed feed.

Modifications to this design have been considered, such as is disclosed in U.S. Pat. No. 5,358,698. The goal of such arrangements is to enhance certain performance and operating characteristics of the catalyst, such as its selectivity and activity. “Selectivity” is the ability of a catalyst to selectively produce higher levels of the desired product and lower levels of undesired products. For example, in the conversion of ethylbenzene to styrene, higher selectivity produces higher levels of styrene and lower levels of various by-products. “Activity” is the ability of a catalyst to convert a certain percentage of the ethylbenzene to styrene during each pass of the feedstock over the catalyst. Higher activity means a higher percentage of the ethylbenzene is converted into styrene.

The conversion of ethylbenzene to styrene is an endothermic reaction which requires the addition of heat to the process to maintain an appropriate level of activity. The rate of flow of the ethylbenzene must be controlled as it passes across the catalyst bed to maintain acceptable selectivity and activity. Thus, the liquid hourly space velocity (LHSV) of the gas stream through the adiabatic reactor may be severely limited.

As time passes, the catalyst loses selectivity and activity. Accordingly, in the past the process for the dehydrogenation of ethylbenzene has required the use of multiple reactors with a limit on the thickness of the catalyst bed in any one reactor. One proposed solution to this problem is disclosed in U.S. Pat. No. 6,096,937.

There are two entirely different recognized types of ethylbenzene dehydrogenation reaction. The conventional dehydrogenation reaction (nonoxidative dehydrogenation) occurs over an iron oxide catalyst and requires that oxygen must not be present in the feed stream. The second type of dehydrogenation reaction is oxidative dehydrogenation where the feed stream contains ethylbenzene and a source of oxygen. That feed stream is passed over an oxidative dehydrogenation catalyst, where an oxidation reaction occurs to generate heat. The reaction is as follows:

C₆H₅CH₂CH₃+½O₂→C₆H₅CH=CH₂+H₂O

Oxidative dehydrogenation is exothermic and irreversible. An oxidative dehydrogenation reaction is disclosed for example in U.S. Pat. Nos. 5,510,553 and 4,777,319. This process is also discussed in Kirk-Othmer, Encyclopedia of Chemical Technologies, Volume 22, page 978 (1996). Additionally, UOP has disclosed a process for oxidatively reheating a reaction mixture including ethylbenzene using a radial reactor system containing two oxidation catalyst beds utilized in combination with three conventional dehydrogenation catalyst beds in U.S. Pat. No. 5,043,500. In the process of this patent an oxidation catalyst bed (64) is physically located in a vertically layered bed next to a dehydrogenation catalyst (66) within the reactor. See also Kirk-Othmer, Encyclopedia of Chemical Technologies, Volume 22, pages 978-980 (1996).

The process of “oxidative” dehydrogenation has not attained commercial acceptance for various reasons, such as the safety of the oxygen mixing step, potential damage to the dehydrogenation catalyst by oxidation and the possibility of contaminating the styrene product with oxygenates.

Conventional “nonoxidative” dehydrogenation reactions also use reactors but conventionally utilize only a single dehydrogenation catalyst, such as a conventional iron oxide catalyst containing a small amount of potassium and chrome as disclosed, for example, in U.S. Pat. Nos. 2,866,790 and 2,866,791. Various catalysts for nonoxidative dehydrogenation are also disclosed in application Ser. No. 09/237,408, the contents of which are incorporated by reference into this application.

Conventional nonoxidative dehydrogenation catalysts in reactors deactivate during normal use, causing a reduction in ethylbenzene conversion. Further, there are limitations on the activity and selectivity of conventional dehydrogenation catalysts. Other problems that may occur when conventional nonoxidative catalysts are utilized in a conventional radial reactor include a susceptibility to CO₂ poisoning, reduced steam to oil stability, shortened life span, pressure drop, loss of stability at reaction pressures and increased contamination.

Therefore, it is an object of this invention to disclose a novel process for the nonoxidative dehydrogenation of an alkylaromatic feed stream in a radial reactor.

It is a further object of this invention to disclose a unique combination of two or more dehydrogenation catalysts combined together in a single radial reactor for the nonoxidative dehydrogenation of an alkylaromatic feed stream, wherein the two dehydrogenation catalysts have different performance and/or operating characteristics.

It is another object of this invention to disclose a novel process for the nonoxidative dehydrogenation of an alkylaromatic feed stream in a radial reactor, wherein two or more nonoxidative dehydrogenation catalysts are placed within a vertically layered catalyst bed within the radial reactor and wherein at least one of the catalysts has a higher activity than at least one of the other nonoxidative dehydrogenation catalysts.

It is another object of this invention to disclose a novel process for the nonoxidative dehydrogenation of an alkylaromatic feed stream in a radial reactor, wherein two or more nonoxidative dehydrogenation catalysts are placed within a vertically layered bed within the radial reactor and wherein at least one of the catalysts has a higher selectivity than at least one of the other nonoxidative dehydrogenation catalysts.

It is another object of this invention to disclose a novel process for the nonoxidative dehydrogenation of an alkylaromatic feed stream in a radial reactor, wherein two or more nonoxidative dehydrogenation catalysts are placed within a vertically layered bed within the radial reactor and wherein at least one of the catalysts has improved stability at lower reaction pressures than at least one of the other nonoxidative dehydrogenation catalysts.

It is another object of this invention to disclose a novel process for the nonoxidative dehydrogenation of an alkylaromatic feed stream in a radial reactor, wherein two or more nonoxidative dehydrogenation catalysts are placed within a vertically layered bed within the radial reactor and wherein at least one of the catalysts produces lower pressure drop than at least one of the other nonoxidative dehydrogenation catalysts.

It is another object of this invention to disclose a novel process for the nonoxidative dehydrogenation of an alkylaromatic feed stream in a radial reactor, wherein two or more nonoxidative dehydrogenation catalysts are placed within a vertically layered bed within the radial reactor and wherein at least one of the catalysts produces improved steam/oil stability than at least one of the other nonoxidative dehydrogenation catalysts.

It is another object of this invention to disclose a novel process for the nonoxidative dehydrogenation of an alkylaromatic feed stream in a radial reactor, wherein two or more nonoxidative dehydrogenation catalysts are placed within a vertically layered bed within the radial reactor and wherein at least one of the catalysts has a longer effective catalyst life at normal reaction temperatures than at least one of the other nonoxidative dehydrogenation catalysts.

It is another object of this invention to disclose a novel process for the nonoxidative dehydrogenation of an alkylaromatic feed stream in a radial reactor, wherein two or more nonoxidative dehydrogenation catalysts are placed within a vertically layered bed within the radial reactor and wherein at least one of the catalysts is produced in a different physical shape than at least one of the other nonoxidative dehydrogenation catalysts to enhance the overall performance of the catalysts.

It is another object of this invention to disclose two or more nonoxidative dehydrogenation catalysts for use in the nonoxidative dehydrogenation of an alkylaromatic feed stream, wherein each of the catalysts is located in a separate, vertical layer within a single radial reactor and where at least one of the catalysts has a different operating or performance characteristic than at least one of the other catalysts.

### SUMMARY OF THE INVENTION

This invention is directed to a process for the nonoxidative dehydrogenation of an alkylaromatic feed stream comprising passing an alkylaromatic feed stream through a radial reactor containing nonoxidative dehydrogenation catalysts, wherein the nonoxidative dehydrogenation catalysts are arranged in a vertically layered bed within the radial reactor, wherein the nonoxidative dehydrogenation catalysts comprise at least a first nonoxidative dehydrogenation catalyst, and a second dehydrogenation catalyst, wherein each of the dehydrogenation catalysts are located in a different layer within the radial reactor and wherein at least one of the nonoxidative dehydrogenation catalysts has a different performance and/or operating characteristic than at least one of the other nonoxidative dehydrogenation catalysts.

The invention is further directed to at least a pair of nonoxidative dehydrogenation catalysts useful for a process for the nonoxidative dehydrogenation of an alkylaromatic feed stream in a radial reactor, wherein each of the catalysts is located in a separate, vertical layer within the radial reactor and wherein at least one of the catalysts has a different operating and/or performance characteristic than at least one of the other catalysts.

### BRIEF DESCRIPTION OF THE DRAWING

FIG. 1 is a schematic view of the reaction vessel for the nonoxidative dehydrogenation of an alkylaromatic feed stream of the invention.

FIG. 2 is a graph comparing the performance of two nonoxidative dehydrogenation catalysts used individually for ethylbenzene conversion with the performance of the two catalysts used in combination.

### DETAILED DESCRIPTION OF THE INVENTION

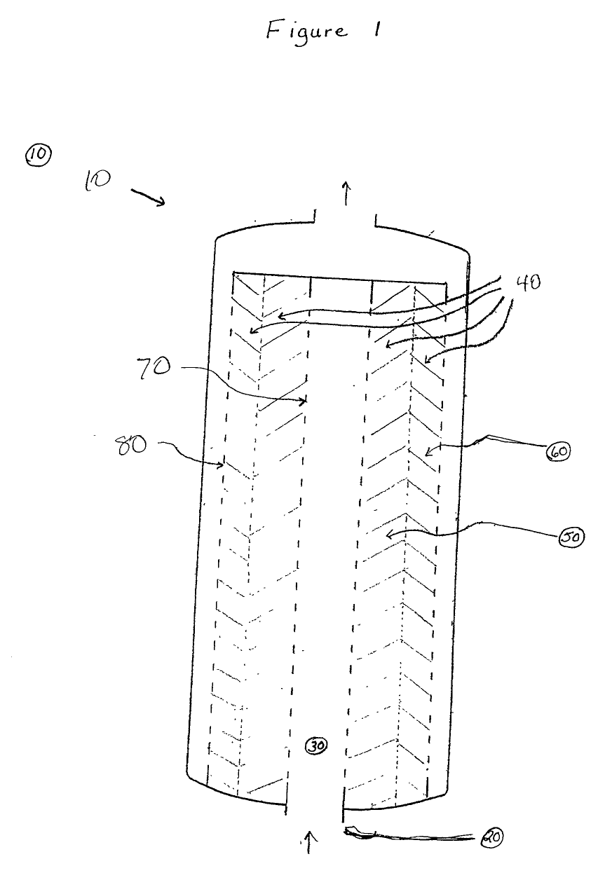

The invention is a process for the nonoxidative dehydrogenation of an alkylaromatic feed stream, wherein the alkylaromatic feed stream is passed through a radial reactor containing two or more nonoxidative dehydrogenation catalysts, at least two of which have different operating and/or performance characteristics. Referring to FIG. 1, which is a cross-sectional schematic drawing showing the radial reactor (10) of the invention, the feed stream, comprising an alkylaromatic and steam, is introduced through an inlet pipe (20) into the center (30) of the radial reactor (10). The alkylaromatic, preferably ethylbenzene, feed stream is heated by mixing it with super heated steam. The heated feed stream is then passed into the radial reactor (10). The admixture enters the inlet pipe (20) of the radial reactor (10) and is radially distributed through the dehydrogenation catalysts bed (40).

The catalysts bed (40) is comprised of two or more layers of nonoxidative dehydrogenation catalysts, at least one of which has a different performance and/or operating characteristic than one of the other nonoxidative dehydrogenation catalysts and wherein the catalysts are arranged in a different vertical layer within the reactor bed (40). (While only a pair of nonoxidative dehydrogenation catalysts are shown in FIG. 1, three or more catalysts are within the scope of the invention as long as at least two of those catalysts have different operating and performance characteristics. In addition, although the preferred embodiment requires that each different catalyst is maintained in a separate bed, it is within the scope of the invention that two or more of the catalysts may be mixed together within a single layer.) Each catalyst is preferably arranged in a separate annular layer within the radial reactor (10) as shown in FIG. 1. Although formal separation of the two catalysts can be effected by use of a device such as a screen, such as is shown in U.S. Pat. No. 5,043,500, in a preferred embodiment the two separate types of nonoxidative dehydrogenation catalysts are in intimate contact with each other within the radial reactor (10). Some mixing of the different catalysts occurs at the boundary line between the catalysts. The catalysts in the catalyst bed (40) are retained in place within the catalyst bed (40) of the radial reactor (10) by use of an inner screen (70) and an outer screen (80), as shown in FIG. 1.

In a preferred embodiment, the catalyst bed (40) contains at least two different nonoxidative dehydrogenation catalysts, wherein at least one of the catalysts has at least one different performance and/or operating characteristic than at least one of the other nonoxidative dehydrogenation catalysts. Many different combinations of dehydrogenation catalysts are within the scope of the invention depending on which performance and/or operating characteristics are desired for the multi-catalyst system.

For example, in one embodiment, one of the nonoxidative dehydrogenation catalysts exhibits higher activity than at least one of the other nonoxidative dehydrogenation catalyst. In this embodiment, a non-oxidative dehydrogenation catalyst which has very high activity, but which is susceptible to CO₂ poisoning, is placed in the outer layer (60) of the catalyst bed (40) and a second, less active nonoxidative dehydrogenation catalyst, which produces relatively low CO₂ amounts, is placed within the inner layer (50) within the radial reactor (10). This type of catalyst layering increases the overall activity of the catalyst system above that which is predicted for the individual activities of the individual nonoxidative dehydrogenation catalysts.

In another embodiment, two separate nonoxidative dehydrogenation catalysts with varying steam/oil stability are placed within the catalyst bed (40) of the radial reactor (10). For example, in this embodiment a nonoxidative dehydrogenation catalyst which has high activity and selectivity, but which has only average steam/oil stability is placed in the outer layer (60) of the catalyst bed (40). A second nonoxidative dehydrogenation catalyst which has superior steam/oil stability but lesser activity and/or selectivity is placed in the inner layer (50) of the vertically layered catalyst bed. The results of such catalyst layering are greater activity and selectivity with enhanced steam/oil stability than is predicted from the performance of the individual nonoxidative dehydrogenation catalysts.

In another embodiment of the invention as shown in FIG. 1, a nonoxidative dehydrogenation catalyst which has exceptional steam/oil stability but which is less tolerant of high temperatures in the reactor (10) is placed in the outer layer (60) of the catalyst bed (40) and a second nonoxidative dehydrogenation catalyst which has good activity and selectivity and which is more resistant to degradation at higher temperatures is placed in the inner layer (50) of the catalyst bed (40). The results of such catalyst layering are good selectivity and activity with longer life for the catalysts than is predicted from the performance of the individual nonoxidative dehydrogenation catalysts.

In a preferred embodiment as shown in FIG. 1, the dehydrogenation catalyst contained in the inner layer (50) is a conventional dehydrogenation catalyst, such as Styromax® catalyst produced by Süd-Chemie Inc., which catalyst is comprised of iron oxide and potassium oxide. The second nonoxidative dehydrogenation catalyst fills the outer layer (60) within the radial reactor (10). This second catalyst has higher selectivity and activity than the nonoxidative dehydrogenation catalyst that is contained within the inner layer (50) of the radial reactor (10). In a preferred embodiment, this catalyst is comprised of iron oxide, an alkali metal source, such as potassium, and one or more promoter. Various types of promoters for conventional nonoxidative dehydrogenation catalysts are known in the art and are included with this disclosure.

In a particularly preferred embodiment the second nonoxidative dehydrogenation catalyst is the catalyst disclosed in Ser. No. 09/143,637, which is incorporated herein by reference. This catalyst is comprised of about 30 to about 90 by weight percent of an iron compound, such as iron oxide, or a ferrite, preferably potassium ferrite, about 1 to about 50 weight percent of an alkali metal source, preferably a sodium or potassium derivative and a source for a noble metal. The source for the noble metal may include elemental noble metal, compounds containing the noble metal or combinations thereof. For purposes of the present invention, the term “noble metal” includes platinum, palladium, rhodium, ruthenium, rhenium, iridium and osmium, with rhodium, ruthenium, platinum and palladium preferable based on performance alone. Based on cost and product availability factors only, palladium and ruthenium are more preferred than platinum, rhodium and rhenium, and to an even greater extent, iridium and osmium are less preferred.

In addition to the above-described components, the preferred second nonoxidative dehydrogenation catalyst may also include as a promoter an oxide or salt of the lanthanides having atomic number of 57 to 62, most preferably cerium. The second nonoxidative dehydrogenation catalyst preferably may also include a molybdenum or tungsten compound, preferably oxides, most preferably molybdenum oxide. The second nonoxidative dehydrogenation catalyst preferably may also include alkaline earth metal compounds, most preferably magnesium oxide or calcium oxide. The second nonoxidative dehydrogenation catalyst may also include a source for titanium, chromium or silicon or aluminum, preferably an oxide or salt. The second nonoxidative dehydrogenation catalyst may also include a source for at least one of the following elements including zinc, manganese, copper, cobalt and vanadium and combinations thereof.

In a more preferred embodiment, the second nonoxidative dehydrogenation catalyst of this invention is composed of about 30 to about 90 weight percent iron oxide calculated as Fe₂O₃, about 1 to about 50 weight percent of an oxide, hydroxide, carbonate, or bicarbonate of an alkali metal, calculated as an oxide and from less than about 1000 ppm of a noble metal, wherein the noble metal is preferably rhodium, platinum, palladium, ruthenium or iridium, wherein said weight percents are based on the total catalyst weight. Preferably the amount of the noble metal present is less than about 300 ppm. For palladium, rhenium and rhodium preferably the amount of metal is less than about 100 ppm, and most preferably less than 20 ppm. Preferably, the second nonoxidative dehydrogenation catalyst also contains as promoters one or more of the following: about 0.5 to about 25 weight percent cerium oxide calculated as CeO₂, from about 0.5 to about 10.0 weight percent molybdenum oxide or tungsten oxide calculated as MoO₃ or WO₃, from about 0.2 to about 10.0 weight percent an alkaline earth metal oxide, preferably magnesium or calcium oxide. Additional components of the second nonoxidative dehydrogenation catalyst may include from about 50 ppm to about 4.0 weight percent of chromium oxide calculated as Cr₂O₃ and from about 10 ppm to about 2000 ppm of titanium oxide calculated as TiO₂. The second nonoxidative dehydrogenation catalyst may also include from about 0.1 to about 10.0 weight percent of the salt or oxide of one or more of the following: aluminum, silicon, zinc, manganese, cobalt, cadmium, vanadium and copper, alone or in combination, calculated on an elemental basis.

A particularly effective second nonoxidative dehydrogenation catalyst contains from about 40 to about 90 weight percent iron oxide calculated as Fe₂O₃, from about 5 to about 50 weight percent of an alkali metal compound calculated as an alkali metal oxide and preferably less than about 300 ppm of a source of a noble metal, wherein the noble metal is preferably palladium, rhenium, rhodium, ruthenium, platinum or iridium. For palladium, rhenium and rhodium preferably the amount of metal present is less than 100 ppm, most preferably less than about 20 ppm. The source for the noble metal is selected from the group including elemental noble metals, preferably elemental palladium, elemental rhodium, elemental ruthenium, elemental platinum and elemental iridium, compounds containing noble metals, preferably compounds containing palladium and/or ruthenium and/or rhodium and/or platinum and/or iridium and combinations thereof. The second nonoxidative dehydrogenation catalyst also preferably includes from about 0.5 to about 10 weight percent of a molybdenum or tungsten compounds calculated as MoO₃ or WO₃, and from about 4 to about 30 weight percent of a cerium compound, calculated as CeO₂, wherein all weight percents are based on the total weight of the catalyst. Additional promoters may be included with this catalyst as discussed above.

A most preferable second nonoxidative dehydrogenation catalyst contains from about 40 to about 90 percent iron oxide calculated as Fe₂O₃, about 5 to about 50 percent of an alkali metal compound, preferably potassium oxide, about 4 to about 30 percent of cerium oxide calculated as CeO₂, about 0.5 to about 10 percent of molybdenum or tungsten oxide calculated as MoO₃ or WO₃, preferably molybdenum oxide, about 0.2 to about 10 percent of calcium or magnesium oxide, preferably calcium oxide, about 10 ppm to about 1000 ppm of titanium oxide calculated as TiO₂, about 100 ppm to about 2000 ppm of chromium oxide calculated as Cr₂O₃, and less than about 20 ppm of a source for a noble metal, wherein the noble metal is preferably palladium, ruthenium, rhodium, platinum and/or iridium, and wherein the percentage is calculated on an elemental basis. Additional components that can be added to this second nonoxidative dehydrogenation catalyst include from about 0.1 to about 10 weight percent of an oxide of aluminum, silicon, manganese, copper, zinc, cadmium, vanadium, and cobalt, calculated on an elemental basis.

This second nonoxidative dehydrogenation catalyst is preferably any one of the catalysts disclosed in Ser. Nos. 09/053,234, 09/143,637 and 09/237,408, each of which is incorporated into this application by reference.

More than two nonoxidative dehydrogenation catalysts may be utilized in this radial reactor (10), each forming a different vertical layer as long as at least one of the catalysts has a different performance and/or operating characteristic than at least one of the other catalysts. Different layers of the same nonoxidative dehydrogenation catalysts may also be sandwiched around nonoxidative dehydrogenation catalysts with different operating or performance characteristics, depending upon the overall performance or operating characteristics that are desired.

In one preferred embodiment of the nonoxidative dehydrogenation process of the invention as shown in FIG. 1, the nonoxidative dehydrogenation catalysts are first added to the radial reactor (10), forming separate vertical layers within the radial reactor (50, 60), wherein one layer of the different nonoxidative dehydrogenation catalysts has a different performance and/or operating characteristic than the adjacent layer of nonoxidative dehydrogenation catalyst. In one preferred embodiment, the outer layer (60) of the bed is comprised of a more active and/or more selective nonoxidative dehydrogenation catalyst than the inner layer (50). In this preferred embodiment, the outer layer (60) comprises from about five (5) to about ninety-five (95) percent of the catalyst bed (40) within the radial reactor (10) This percentage is based on the percentage of the horizontal linear bed depth when measured from the inside of the inner layer (50) to the outside of the outer layer (60). In this preferred embodiment the second nonoxidative dehydrogenation catalyst is more active than the first nonoxidative dehydrogenation catalyst. In a further preferred embodiment, the second nonoxidative catalyst is more selective than the first nonoxidative dehydrogenation catalyst.

After the two or more layers of nonoxidative dehydrogenation catalysts are added to the radial reactor, the feed stream comprising the alkylaromatic and steam is then passed through the radial reactor (10).

When using this process, depending on the choice of the operating and/or performance characteristics of the catalysts, there is improved performance, i.e., higher conversion, and improved yield than when a single conventional nonoxidative dehydrogenation catalyst is utilized. Alternatively, when the catalysts that are chosen have different susceptibility to CO₂ poisoning, the results of the catalyst layering are increased activity above that predicted by the individual activities of the individual catalysts. Further, where the catalysts that are chosen have different steam/oil stability, the catalysts exhibit good activity and selectivity with improved steam/oil stability. Further, when the performance or operating characteristics which differ between the two catalysts relate to degradation depending upon temperature, the life of the catalyst bed is extended. Further, depending upon the choice of the physical size of the respective catalysts and the shape of those catalysts, an improvement in pressure drop is experienced. For example, when a smaller size nonoxidative dehydrogenation catalyst (such as 3.0 mm in diameter) is used in the inner layer (50) and a larger size nonoxidative dehydrogenation catalyst (such as 4.5 mm in diameter) is used in the outer layer (60), a reduced pressure drop is obtained without loss of selectivity or activity.

### EXAMPLE

The scheme of the invention was simulated in the laboratory. Rather than a radial reactor, a 1″ id, 100 cc, down flow tubular reactor was prepared. Conditions for the reactor were essentially isothermal with only very small gradients of temperature and pressure across the length of the reactor. A 50/50 (by volume) layering of Catalyst A, the highly active, CO₂ sensitive catalyst, and Catalyst B, the highly selective, low CO₂ producing catalyst were charged to the reactor. Catalyst A was placed at the bottom (outer position) of the down flow reactor. Catalyst B was placed in the top (inner position). Reaction conditions were 1.0 LHSV, 2.0 S/O and 1 atm. pressure. Performance of the layered catalyst set-up was determined at 1150, 1100, 1050 and 1000° F. For comparison, tests identical to that described above were performed on Catalysts A and B separately.

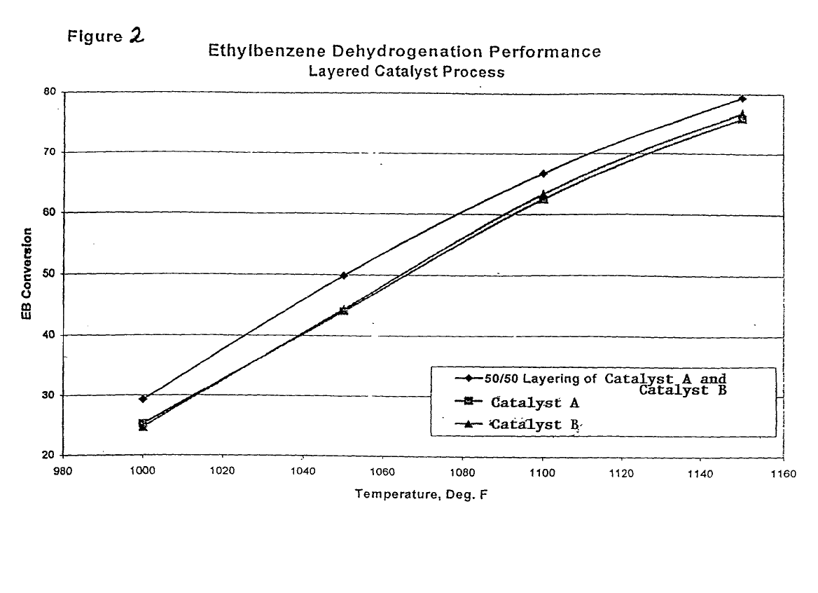

Ethylbenzene conversions versus reaction temperatures of all three tests are shown in FIG. 2. Surprisingly, ethylbenzene conversions from the layered set-up were higher than were obtained with either of the catalysts when tested individually.

Depending on the desired choice of enhanced performance and/or operating characteristics, various layers of different nonoxidative dehydrogenation catalysts can be combined. These combinations may result in enhanced activity and/or selectivity, enhanced steam/oil stability, longer life, reduced pressure drop and/or improved stability at lower reaction temperatures or pressures. It has been surprisingly discovered that the enhancement in performance and/or operating characteristics of the combination of nonoxidative dehydrogenation catalysts exceeds the performance of the individual nonoxidative dehydrogenation catalysts that are combined when those catalysts are used separately.

The principles, preferred embodiments and modes of operation of the present invention have been described in the foregoing specification. The invention which is intended to be protected herein, however, is not to be construed as limited to the particular forms disclosed, since these are to be regarded as illustrative rather than restrictive. Variations and changes may be made by those skilled in the art without departing from the spirit of the invention.

1. A process for the nonoxidative dehydrogenation of an alkylaromatic feed stream comprising passing the alkylaromatic feed stream through a radial reactor containing two or more nonoxidative dehydrogenation catalysts, wherein the nonoxidative dehydrogenation catalysts are arranged in vertically layered beds within the radial reactor, wherein the nonoxidative dehydrogenation catalysts comprise at least a first nonoxidative dehydrogenation catalyst and a second nonoxidative dehydrogenation catalyst, wherein the first and second nonoxidative dehydrogenation catalysts each have different operating or performance characteristics.

2. The catalyst of claim 1 wherein the first nonoxidative dehydrogenation catalyst is placed in an inner, vertical layer within the radial reactor and wherein the second nonoxidative dehydrogenation catalyst is placed within a vertical layer that is a greater linear distance from a center of the radial reactor than is an outer edge of the inner vertical layer.

3. The process of claim 1 wherein one of the nonoxidative dehydrogenation catalysts has a higher selectivity than the other nonoxidative dehydrogenation catalyst.

4. The process of claim 1 wherein one of the nonoxidative dehydrogenation catalysts has a higher activity than the other nonoxidative dehydrogenation catalyst.

5. The process of claim 1 wherein one of the nonoxidative dehydrogenation catalysts has both higher activity and selectivity than the other nonoxidative dehydrogenation catalyst.

6. The process of claim 1 wherein one of the nonoxidative dehydrogenation catalysts has improved stability at lower reaction pressures than does the other nonoxidative dehydrogenation catalyst.

7. The process of claim 1 wherein one of the nonoxidative dehydrogenation catalysts produces a lower pressure drop than does the other nonoxidative dehydrogenation catalyst.

8. The process of claim 1 wherein one of the nonoxidative dehydrogenation catalysts has improved steam/oil stability at a lower reaction temperature than does the other nonoxidative dehydrogenation catalyst.

9. The process of claim 1 wherein one of the nonoxidative dehydrogenation catalysts has a longer effective catalyst life at reaction temperatures than does the other nonoxidative dehydrogenation catalyst.

10. The process of claim 1 wherein one of the nonoxidative dehydrogenation catalysts has a different physical shape than does the other nonoxidative dehydrogenation catalyst.

11. The process of claim 1 wherein one of the nonoxidative dehydrogenation catalysts comprises from about 30 to about 90 weight percent of an iron compound calculated as Fe₂O₃, and about 1 to about 50 weight percent of an alkyl metal source calculated as an alkali metal oxide.

12. The process of claim 11 wherein the nonoxidative dehydrogenation catalyst further comprise one or more promoters.

13. The process of claim 1 wherein the nonoxidative dehydrogenation catalysts comprise a first, second and a third nonoxidative dehydrogenation catalysts, wherein at least one of these catalysts has different performance and/or operating characteristics than at least one of the other two catalysts.

14. The process of claim 1 wherein the nonoxidative dehydrogenation catalysts comprise a first, second, third and fourth nonoxidative dehydrogenation catalysts, wherein at least one of these catalysts has different performance and/or operating characteristics than at least one of the other three catalysts.

15. The process of claim 1 wherein the second nonoxidative dehydrogenation catalyst comprises about 30 to about 90 weight percent of an iron compound calculated as an Fe₂O₃, about 1 to about 50 weight percent of an alkali metal source calculated as an alkali metal oxide, and about 0.1 ppm to about 1000 ppm of a noble metal source selected from the group consisting of elemental noble metals, compounds containing noble metals and combinations thereof, wherein all weight percents are based on the total weight of the catalyst.

16. The process of claim 1 wherein the second nonoxidative dehydrogenation catalyst comprises from about 40 to about 90 weight percent iron oxide calculated as Fe₂O₃, from about 5 to about 50 weight percent of an alkali metal compound calculated as an alkali metal oxide, from about 1 ppm to about 100 ppm of a source of noble metal source selected from the group consisting of an elemental noble metals, compounds containing a noble metal and combinations thereof, from about 0.5 to about 10 weight percent of a molybdenum or tungsten compound, calculated as MoO₃ or WO₃, and from about 4 to about 30 weight percent of a cerium compound, calculated as CeO₂, wherein all weight percents are based on the total weight of the catalyst.

17. The process of claim 1 wherein the second nonoxidative dehydrogenation catalyst comprises from about 40 to about 90 weight percent iron oxide calculated as Fe₂O₃, from about 5 to about 50 percent of a potassium compound calculated as potassium oxide, from about 0.1 ppm to about 20 ppm of a noble metal source selected from the group consisting of an elemental noble metal, compounds containing a noble metal and combinations thereof, from about 0.5 to about 10 weight percent of a molybdenum or tungsten compound calculated as MoO₃ or WO₃, from about 4 to about 30 weight percent of a cerium compound calculated as CeO₂, from about 0.2 to about 10 weight percent of a calcium or magnesium compound calculated as an oxide, from about 100 ppm to about 2000 ppm of a chromium compound calculated as Cr₂O₃, and from about 10 ppm to about 1000 ppm of a source for titanium calculated as TiO₂, wherein all weight percents are based on the total weight of the catalyst.

18. The process of claim 1 wherein the first nonoxidative dehydrogenation catalyst comprises about 5 to about 95 percent of the nonoxidative dehydrogenation catalysts contained within the radial reactor, wherein the percentage is based on the linear bed depth measurement of the catalysts within the reactor.

19. The process of claim 1 wherein the first and second nonoxidative dehydrogenation catalysts are in intimate contact with each other within the radial reactor.

20. The process of claim 1 wherein the alkylaromatic feed stream comprises ethylbenzene and steam.
